(19) United States
(12) Patent Application Publication
(10) Pub. No.: US 2026/0094691 A1
(43) Pub. Date: Apr. 2, 2026

(54) ERROR CORRECTION WITH EMBEDDED PARITY

(71) Applicant: GLOBUS MEDICAL, INC., AUDUBON, PA (US)

(72) Inventor: Sanjay M. Joshi, Andover, MA (US)

(21) Appl. No.: 18/903,032

(22) Filed: Oct. 1, 2024

(51) Int. Cl. G16H 30/20; G06T 11/00

(52) U.S. Cl. CPC G16H 30/20; G06T 12/10

(57) ABSTRACT

Methods and data structures provide protection for a set of digital image files from loss, wherein the set of digital image files comprises a plurality of digital image files of a same size. Methods include encoding each digital image file in the plurality thereof, encoding the resulting check blocks by computing a final check symbol, and embedding the check symbols and final check symbols into the plurality of digital image files in a manner distributed approximately evenly over the plurality of digital image files. The digital image files may be transmitted to a receiver, and decoded, including detaching the check symbols and final check symbols, creating a plurality of error correction blocks from the same, and using these to recover or repair any errors in the received digital image files.

### BACKGROUND

The present disclosure relates to error correction in data transmission. More particularly, the disclosure relates to methods and data structures for protecting a set of digital images from loss.

Forward error correction (FEC) in data transmission is a well-studied technique in information theory for repairing data that is lost or corrupted in transmission of the message. In FEC, one or more encoders are used to compute a number of check symbols from the message symbols to be transmitted. These redundant symbols are transmitted with the message. The corresponding decoders at the receiver process the message plus the check symbols, and these check symbols may be used to recover or repair any incorrect or missing symbols, without the need for a second channel of communication back to the sender.

Traditionally, the message symbols may be divided into a number of message blocks, and the check symbols may be sent as additional block(s) which are transmitted in addition to the message blocks. However, the transmission of additional block(s) containing the check symbols may not be practical in all applications, including, e.g., in the transmission of three-dimensional (3D) diagnostic imaging scans such as, e.g., computed tomography (CT) or magnetic resonance imaging (MRI) scans. For example, the receiver may be expecting a certain number of blocks of symbols, and may not be configured to process the additional blocks containing the check symbols.

Digital Imaging and Communications in Medicine (DICOM) is the industry standard format for producing, storing, displaying, sending, querying, processing, retrieving, and printing medical images including, e.g., 3D diagnostic imaging scans such as, e.g., CT or MRI scans. In this format, the images are stored as binary files with header and pixel values, and are typically archived in a Picture Archive and Communication System (PACS).

For a 3D scan such as CT or MRI, the image pixels are stored as a quantity of two-dimensional (2D) slices of a volume, each 2D slice in a different file. Each 2D slice includes a block of pixel data, typically having 16-bits per pixel, and a DICOM header. The files may be delivered on a USB drive or an optical disc such as a CD or DVD. If the delivery medium is corrupted (e.g., the CD or DVD is scratched), one or more 2D slices in a 3D volume may be lost. Depending on the anatomy covered by these lost or corrupted 2D slices, the entire 3D volume may become unusable. Getting a second copy of the data may be impossible or impractical, especially if the need for the data is immediate, such as inside an operating room.

### BRIEF DESCRIPTION OF THE INVENTION

A first aspect of the disclosure provides a method for protecting a set of digital image files from loss, wherein the set of digital image files comprises a plurality of digital image files of a same size, the method comprising encoding each digital image file in the plurality of digital image files by computing a plurality of check symbols for a plurality of symbols contained in a same location in each digital image file; and generating a plurality of check blocks, each check block in the plurality thereof including a quantity of check symbols equal to a number of symbols contained in the digital image file. The method further includes encoding each check block in the plurality thereof by computing a final check symbol therefor; and embedding the check symbols and the final check symbols into the plurality of digital image files, wherein the check symbols and the final check symbols are distributed approximately evenly over the plurality of digital image files.

In certain embodiments, after embedding, the method includes transmitting the set of digital image files to a receiver.

In certain embodiments, after transmitting, the method includes decoding each digital image file in the received set of digital image files by detaching the check symbols and the final check symbols from the plurality of digital image files; and creating a plurality of error-correction blocks using the detached check symbols and final check symbols; and performing error-correction by recovering or repairing one or more received error-correction blocks; and using the recovered or repaired error-correction blocks to recover or repair received digital image files.

In certain embodiments, after embedding, the method includes encoding each digital image file having the check symbol embedded therein, to add a secondary check symbol within each digital image file. In further embodiments, after the detaching, and prior to the creating, the method includes decoding the secondary check symbol within each digital image file.

In certain embodiments, embedding comprises an iterative series of embedding operations.

In certain embodiments, each digital image file in the plurality thereof comprises Digital Imaging and Communications in Medicine (DICOM) formatting.

In certain embodiments, each digital image file in the plurality thereof comprises a block of pixel data and a DICOM header, and the check symbols and the final check symbol are embedded into the DICOM header as metadata.

In certain embodiments, the plurality of digital image files comprises a series of portable network graphics (PNG) image files, and the check symbols and the final check symbol are embedded into the PNG image files as private blocks.

In certain embodiments, the set of digital image files are adapted for storage, transmission, writing, or archiving in a Picture Archive and Communication System (PACS).

In certain embodiments, the set of digital image files represents a computed tomography (CT) scan or a magnetic resonance imaging (MRI) scan.

A second aspect of the disclosure provides a data structure stored on a computer readable medium, comprising a set of data sub-structures, wherein each data sub-structure corresponds to a digital image, and the set of digital images collectively corresponds to a three-dimensional (3D) scanned volume, wherein each data sub-structure in the set thereof comprises a first data field containing a series of image data; and a second data field containing a plurality of check symbols computed based on the series of image data contained in the first data field, wherein a quantity of check symbols contained in the second data field is equal to a quantity of image data symbols contained in the first data field, and wherein the plurality of check symbols are adapted for decoding by a processor to protect the data structure from loss in transmission.

In certain embodiments, each data sub-structure includes Digital Imaging and Communications in Medicines (DICOM) formatting, wherein the second data field comprises a custom tag in a DICOM header.

In certain embodiments, each data sub-structure includes Portable Network Graphics (PNG) formatting, wherein the second data field comprises a private block.

In certain embodiments, the series of image data comprises a plurality of blocks of pixel data.

In certain embodiments, the pixel data comprises 16 bits per pixel.

In certain embodiments, the data structure is adapted for archival in a Picture Archive and Communication System (PACS).

In certain embodiments, the digital image is a two-dimensional (2D) slice of a three-dimensional (3D) diagnostic imaging scan, and the 3D diagnostic imaging scan comprises a computed tomography (CT) or magnetic resonance imaging (MRI) scan.

A third aspect of the disclosure provides a method of reconstructing a digital image file in a set of digital image files, wherein the set of digital image files comprises a plurality of check symbols and final check symbols embedded therein, the method comprising detaching the check symbols and the final check symbols from the set of digital image files; creating a plurality of error-correction blocks using the detached check symbols and final check symbols; and reconstructing the digital image file by recovering or repairing one or more error-correction blocks; and using the recovered or repaired error-correction blocks to reconstruct the digital image file.

In certain embodiments, the set of digital image files represents a computed tomography (CT) scan or a magnetic resonance imaging (MRI) scan.

These and other aspects, advantages and salient features of the invention will become apparent from the following detailed description, which, when taken in conjunction with the annexed drawings, where like parts are designated by like reference characters throughout the drawings, disclose embodiments of the invention.

### BRIEF DESCRIPTION OF THE DRAWINGS

FIG. 1 illustrates an example of a message having eight message blocks and a checksum block, in accordance with some embodiments.

FIG. 2 illustrates an example of a message having eight message blocks, and a checksum distributed across the first seven message blocks, in accordance with some embodiments.

FIG. 3 illustrates a flow diagram for error correction with embedded parity, in accordance with some embodiments.

FIGS. 4A and 4B illustrate flow diagrams for single step and iterative coding of check symbols, respectively, during the EncodeC process in FIG. 3, in accordance with some embodiments.

FIG. 5 illustrates a flow diagram for adding check symbols within blocks, in accordance with some embodiments.

FIG. 6 illustrates a block diagram of a system for acquiring, storing, transmitting, and receiving 3D diagnostic imaging scans, in accordance with some embodiments.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION OF THE INVENTION

Various embodiments of the present disclosure are described below in reference to their uses in methods and data structures for protecting digital image files, such as sets of 2D digital image files corresponding to a 3D diagnostic imaging scan, from loss due to, e.g., transmission errors or corruption. According to embodiments of the disclosure, forward error correction (FEC) techniques are used to create redundant check symbols which may be embedded within each block of message symbols in such a way that the number of blocks of symbols stays the same as in the original message.

Practical applications include self-correction of missing or corrupt slices from a 3D diagnostic imaging scan such as a CT or MRI scan. A need exists for methods and data structures for the protection of digital image files, such as those making up 3D diagnostic scans, against such losses. Advantageously, such methods and data structures may be capable of outputting FEC check blocks as DICOM data, enabling a PACS to store, transmit, and write the files. Further advantageously, the methods and data structures may avoid the need to separately export these additional check blocks, which may not be rendered as medical images on a PACS, to a recordable storage medium such as a USB or CD/DVD together with the digital image files themselves.

As used herein, the term “digital image file” is used interchangeably with, and is considered equivalent to and synonymous with the terms “digital image” and “image file.”

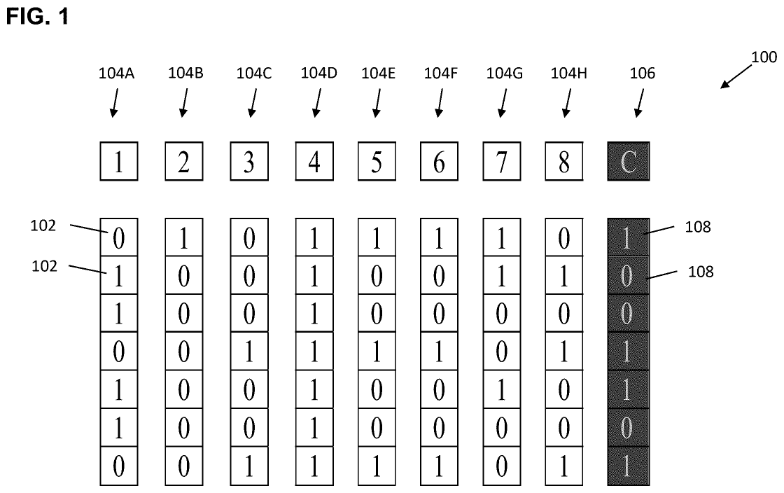

Turning first to FIG. 1, a simplified example of performing forward error correction (FEC) on an image file is illustrated. A message 100 may contain a plurality of message blocks 104 (104A . . . 104H), each of which contains a plurality of message symbols 102. In the example of FIG. 1, the message 100 includes eight (8) message blocks 104A . . . 104H, each of which contains seven (7) message symbols 102. The example message 100 includes fifty-six (56) message symbols 102, which may include bits (0, 1), spread over eight (8) blocks 104A . . . 104H.

The check block (or checksum block) 106 contains parity check bits, or check symbols 108, which are computed as ‘0’ for an even number of ‘1’ bits in the message, and ‘1’ otherwise, e.g., for an odd number of ‘1’ bits in the message. The check block 106 is computed, including all check symbols 108, and transmitted with the message to a receiver. The check symbols 108 may be computed using any of a number of techniques. These may include, e.g., concatenated error correcting codes (ECCs), low-density parity-check (LDPC), turbo codes, local decoding and testing of codes, and interleaving. In a simplified example, it may be assumed that by encoding a message 100 of length k with check of length t, it is possible for a decoder to correct up to t missing message symbols 102 at known locations in the message 100. The mathematical convention for this scheme is (n, k) code, where n=k+t.

In the event that the receiver does not receive one of the message blocks, e.g., if message block 104H is not received, the receiver can compute the lost message symbols 102 contained within the lost message block 104H using the check block 106. A lost message symbol 102 may be 0 if the parity of the rest of the symbols 102 matches the corresponding check symbol 108 in the check block 106. Alternatively, the lost message symbol 102 may be 1 if the parity of the rest of the symbols 102 does not match the corresponding check symbol 108 in the check block 106. This may be repeated for each message symbol 102 in the message block 104H, facilitating recovery of all data contained in the message block 104H.

In certain applications, transmission of the redundant check block 106 shown in FIG. 1 may be impractical. For example, a PACS (Picture Archive and Communication System) for managing DICOM-formatted medical imaging files may not be configured to process a 3D imaging scan file containing a different quantity of DICOM files than expected. Additionally, DICOM-formatted digital image files are typically stored as binary files with header and pixel values. While check blocks 106 may be formatted as DICOM data so that the PACS can store, transmit, and write them, these additional check blocks 106, which may not be rendered as medical images on PACS, would need to be exported or written to a recordable medium (e.g.

USB drive or optical disc such as a CD or DVD) together with the message blocks. This creates an extra step that a user may or may not understand or be aware of when transmitting 3D scan files.

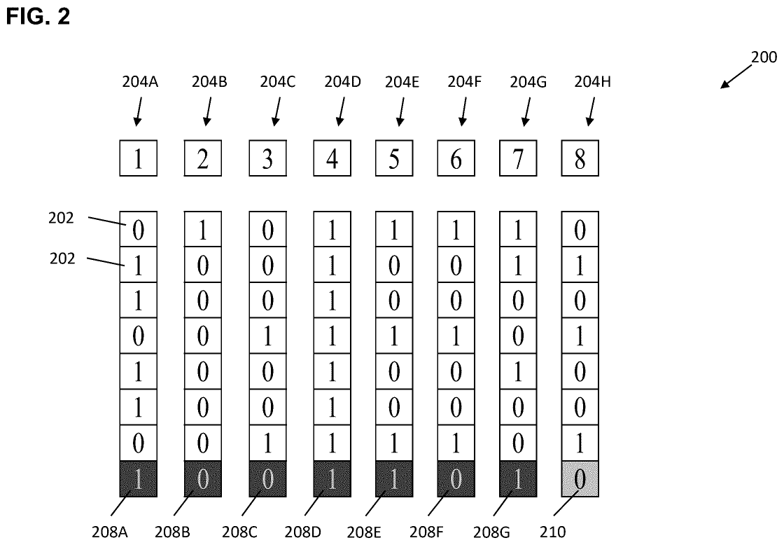

Turning to FIG. 2, a method is provided according to an embodiment of the disclosure for protecting a set of message blocks 204, corresponding to digital image files, from loss. In FIG. 2, a message 200 may contain a plurality of message blocks 204 (204A . . . 204H), each of which contains a plurality of message symbols 202. In the example of FIG. 2, the message 200 includes eight (8) message blocks 204A . . . 204H, each of which contains seven (7) message symbols 202. The example message 200 includes fifty-six (56) message symbols 202, which may include bits (0, 1), spread over eight (8) message blocks 204A . . . 204H.

The message 200 is encoded to yield a check block (or checksum block) containing parity, or check symbols 208. In one example, the check symbols 208 may be computed as, e.g., ‘0’ for an even number of ‘1’ bits in the message, and ‘1’ otherwise, e.g., for an odd number of ‘1’ bits in the message. The check symbols 208A . . . 208G may then be appended to each of message blocks 204A . . . 204G. In this example, the checksum includes seven (7) bits, and is spread across the first seven message blocks 204A . . . 204G. An additional check symbol 210 is computed for the seven-symbol check data (208A . . . 208G) in the same manner as check symbols 208 were computed based on the message symbols 202. The additional check symbol 210 may then be embedded in message block 204H.

Some data storage formats with headers support storing additional ‘custom’ information or meta-data about the message they are carrying. For example, DICOM supports the use of private fields or tags, and PNG (Portable Network Graphics) formatting supports the use of private blocks. In certain embodiments, the check symbols 208 and additional check symbol(s) 210 may be stored within these custom fields or tags.

Thus encoded, the message 200 may then be transmitted to a receiver. The receiver of message 200 receives exactly eight (8) message blocks 204A . . . 204H, which corresponds to the expected number of message blocks 204. Relative to message 100 (FIG. 1), computation and transmission of only one additional symbol (64 symbols in FIG. 2 as compared to 63 symbols in FIG. 1) facilitates the maintenance of the original number of message blocks 204 in the message 200 while including the check symbols 208 and additional check symbol(s) 210. The additional check symbol 210 may be used to facilitate recovery of one of the check symbols 208A . . . 208G in the event that one of the message blocks 204 is not successfully received at the receiver. Upon recovery of the missing check symbol 208, the missing message symbol(s) 202 may also be recovered.

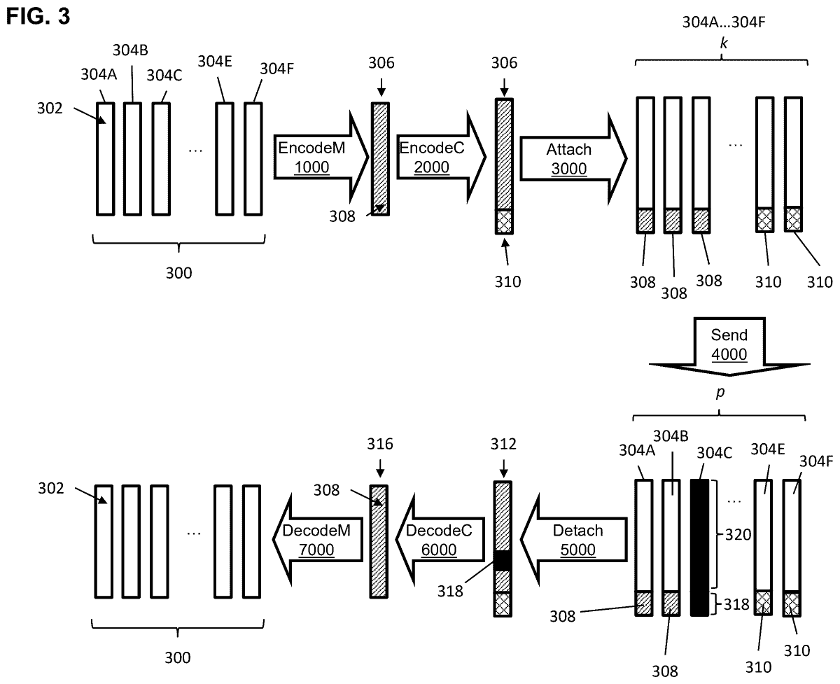

In the example of FIG. 2, the number of check symbols 208 (i.e., seven) plus the number of additional check symbols 210 (i.e., one) equals a total (i.e., eight) that is equal to the number of message blocks 204. This may not be the case in all instances. Accordingly, a more generic algorithm for error correction with embedded parity is described with reference to FIG. 3.

FIG. 3 provides a process flow diagram illustrating a method for protecting a set 300 of digital image files 304 from loss. The set 300 of digital image files may include a plurality of digital image files having a same or common size. For example, the set 300 of digital image files may collectively contain a computed tomography (CT) scan or a magnetic resonance imaging (MRI) scan, in which each digital image file corresponds to a 2D slice of a scanned 3D volume. In certain embodiments, each of the digital image files may correspond to a block of pixel data, e.g., 16 bits/pixel, and may include a DICOM header. In certain embodiments, each digital image file may correspond to a PNG image in a series thereof. Each digital image file may further correspond to a message block 304. The quantity of message blocks 304 in set 300 may be represented by k, and each message block 304 may be made up of m quantity of symbols 302 which are analogous to message symbols 102, 202 in FIGS. 1 and 2, respectively.

Process 1000 (“EncodeM” or “Encode Message”) includes encoding each message block 304A . . . 304F, e.g., each digital image file in the set of digital image files. The encoding may include computing, e.g., with a (k+t, k) code encoder, a plurality of check symbols 308 for a plurality (m) of message symbols 302 contained in a same location in each digital image file or message block 304A . . . 304F. The encoding further includes generating a plurality of check blocks 306, where each check block 306 in the plurality of check blocks 306 includes a quantity of check symbols 308 contained therein that is equal to the number of symbols 302 contained in the message block 304. For each index of the m symbols 302 across k message blocks 304, the (k+t, k) code encoder may compute t check symbols 308. At the end of this operation, the check blocks 306 in the plurality thereof will also have m symbols 308.

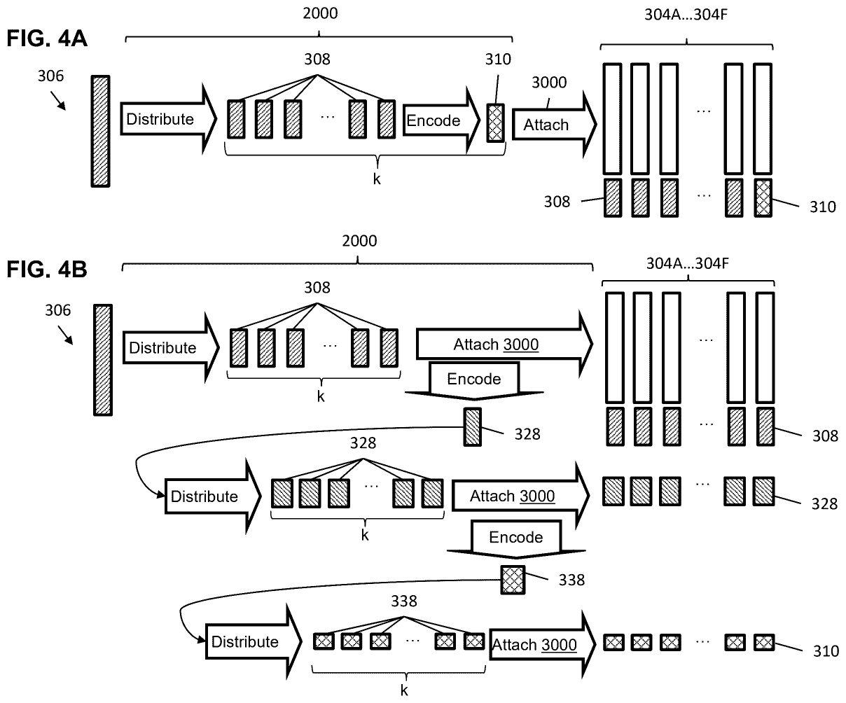

Process 2000 (“EncodeC” or “Encode Check”) includes encoding each check block 306 in the plurality thereof, by computing a final check symbol 310 for the check block 306. The quantity of check symbols to be embedded in the message blocks 304A . . . 304F is (mt). The check symbols provide redundancy as described relative to FIG. 2 when distributed across k quantity of message blocks 304. This may be performed in single-step operation (as shown in FIG. 4A) or an iterative series of operations (as shown in FIG. 4B) as described further below. As compared to a single step operation, an iterative series of operations provides additional redundant symbols, thereby increasing the ability to recover or repair missing or corrupted data. The tradeoff for this increased redundancy is an increase in file size to be transmitted and received.

Process 3000 (“Attach”) includes embedding the check symbols 308 (contained in check block 306) and the final check symbols 310 into the plurality of message blocks 304A . . . 304F, e.g., digital image files. The check symbols 308 and the final check symbols 310 are distributed in approximately even quantities over the plurality of (k) message blocks 304A . . . 304F, in a manner similar to that described relative to check symbols 208A . . . 208G and additional check symbol 210 in FIG. 2. The message blocks 304A . . . 304F with embedded check symbols 308 and final check symbols 310 are ready for transmission.

Process 4000 (“Send”) includes transmitting the set of message blocks 304A . . . 304F, e.g., digital image files, to a receiver. In particular, message blocks 304A . . . 304F of quantity (k) are transmitted with their respective embedded check symbols 308 and final check symbols 310. Up to (k) quantity of message blocks of data may be received at the receiver. Generally, the quantity of (k−t) is less than or equal to the quantity (p) message blocks received at the receiver, such that (p) is less than or equal to the quantity (k). In certain embodiments, all of the quantity (k) message blocks 304A . . . 304F may be received at the receiver, such that the quantity (k) message blocks 304A . . . 304F transmitted is equal to the quantity (p) message blocks 304A . . . 304F received. In this instance, no repair or recovery is required. In other embodiments, one or more of the quantity (k) message blocks 304 may be lost in transmission or corrupted. For example, in the example of FIG. 3, the message block 304C is lost in transmission, such that (p)=(k−1). Upon receipt by the receiver, where (p) is less than (k), the method includes decoding each message block, e.g. digital image file in the received set of digital files, to recover the data contained in the missing message block 304C as described herein.

Process 5000 (“Detach”) includes detaching and collecting the check symbols 308 and the final check symbols 310 from the plurality of correctly received message blocks 304A . . . 304F, e.g., the plurality of digital image files, to create a plurality of error-correction blocks 312. In the example shown in FIG. 3, check symbols 308 are detached from the received message blocks 304A and 304B, and final check symbols 310 are detached from the received message blocks 304E and 304F. The check symbol 308 that was embedded in transmitted message block 304C, which was not received at the receiver, is unable to be included in the error correction block 312. The corrupted and/or missing check symbol is notated with reference 318.

Process 6000 (“DecodeC” or “Decode Check”) includes recovering or repairing one or more receive error-correction blocks 312 created in Process 5000 by the collection of detached check symbols 308 and final check symbols 310, e.g., those collected from received message blocks 304A, 304B, 304E, and 304F. The repair or recovery may be performed using a decoder corresponding to the check block encoder used in process 2000. The decoder computes a quantity (mt) of check symbols contained in the recovered or repaired error correction blocks 316. In this manner, the missing check symbol 318 is repaired or recovered.

Process 7000 (“DecodeM” or “Decode Message”) includes using the recovered or repaired error-correction blocks 316 to recover or repair missing message symbols 302, e.g., those contained in message block 304C. This may be performed, e.g., using the (k+t, k) decoder corresponding to the (k+t, k) encoder used in process 1000, to compute all (mk) message symbols. The (k+t, k) code decoder can successfully recover the (k−p) lost message symbols based on the known locations of lost data, e.g., the lost message symbols 320 from message block 304C. This enables repair or recovery of all message blocks 304 in the message, e.g., set 300 of digital image files.

In some embodiments, with reference to FIG. 4A, process 2000 (“EncodeC” or “Encode Check”) may be performed as a single encoding and embedding operation. In the single encoding operation, the check symbols 308 in check block 306 are distributed into (k−1) quantity of data blocks. A final check symbol 310 is computed based on the check symbols 308, and the (k−1) check symbols 308 and the final check symbol 310 are embedded in the (k) message blocks 304A . . . 304F.

In other embodiments, with reference to FIG. 4B, process 2000 (“EncodeC” or “Encode Check”) may be performed as an iterative series of encoding and embedding operations. In such embodiments, the check symbols 308 in check block 306 are distributed into (k) quantity of data blocks and embedded in the message blocks 304A . . . 304F. Check symbols 308 may be indexed, and the resulting index 328 may be used as an input for a subsequent iterative operation in the series. Index 328 may be distributed into (k) quantity blocks of data and embedded in the message blocks 304A . . . 304F. Index 328 may then itself be indexed to return a final index 338 containing final check symbols 310, including the additional redundant symbols. Final index 338 may be distributed into (k) quantity of blocks of data and embedded into the message blocks 304A . . . 304F for transmission. In the example depicted in FIG. 4B, three iterations of encoding and embedding are depicted, however any number of iterations may be deployed. The greater the number of iterations performed, the larger the resulting message block size will be for transmission and the greater bandwidth that will be required. However, larger numbers of iterations will provide greater redundancy and therefore greater ability to detect and repair or recover errors in transmission. Smaller numbers of iterations, and particularly the single iteration embodiment of FIG. 4A will result in smaller message blocks for transmission, less required storage volume, and lower bandwidth requirements for transmission. However, this approach will provide less redundancy and a comparatively limited ability to detect and repair or recover errors in transmission.

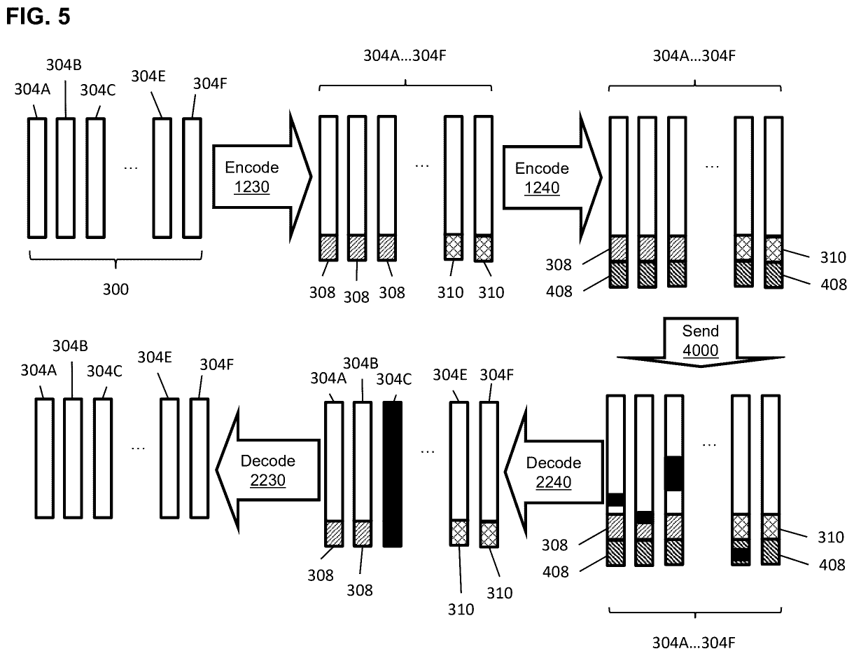

With reference to FIG. 5, a further extension of the foregoing process of FIG. 3 provides additional protection via an additional encoder-decoder operation which can be added to each message block. While more computationally intensive, this addition allows detection and recovery of corrupted data to a further augmented extent.

As shown in FIG. 5, message blocks 304A . . . 304F in the set 300 of message blocks are encoded at Process 1230. Process 1230 includes performing each of processes 1000 (Encode Message), 2000 (Encode Check), and 3000 (Attach) as described relative to FIG. 3, resulting in a set 300 of message blocks 304A . . . 304F having check symbols 308 and final check symbols 310 embedded therein.

After process 1230 concludes with embedding as performed in the manner described relative to process 3000, and prior to transmitting the message 300 at process 4000, each message block 304A . . . 304F, e.g., each digital image file, having a check symbol 308, 310 embedded therein, may be encoded at process 1240 to add a secondary check symbol 408 within each message block 304A . . . 304F. The secondary check symbols 408 are computed within each block, e.g., within message block 304A, rather than across message blocks 304A . . . 304F, and attach to the same message block, e.g., 304A.

Process 4000 (“Send”) includes transmitting the set of message blocks 304A . . . 304F, e.g., digital image files, to a receiver. In embodiments in which one or more message blocks 304 are lost in transmission or corrupted, the method includes decoding each message block 304, e.g. digital image file in the received set of digital files, to recover the data contained in the missing message block 304C as described herein.

Process 2240 includes collecting the secondary check symbols 408 from each of the message blocks 304, and decoding the secondary check symbols 408 within each message block 304. In the example of FIG. 5, this process yields received message blocks 304A, 304B, . . . 304E, and 304F, and identifies message block 304C as lost or corrupted.

Process 2230 includes performing the steps of each of processes 5000 (“Detach”), 6000 (“Decode Check”), and 7000 (“Decode Message”) as described above relative to FIG. 3. Process 2230 yields repaired and recovered message blocks 304A . . . 304F.

As mentioned above, in various embodiments each message block 304A . . . 304F may be a digital image file. The digital image files may correspond to 2D slices of a 3D scanned volume, such that a set of 2D slices collectively make up a 3D scanned volume acquired using, e.g., MRI or CT scanning techniques. In certain embodiments, a 3D scan such as a CT scan may include 128, 160, 192, 256, 320, or 640 2D slice files. In certain embodiments, the digital image files may each include DICOM formatting, and may particularly include a block of pixel data and a DICOM header. In such embodiments, the check symbols and final check symbols may be embedded into the DICOM header as metadata in a private field or tag. In certain embodiments, the digital image files may be a series of PNG image files which collectively make up a 3D scanned volume. In such embodiments, the check symbols and final check symbols may be embedded in the PNG-formatted digital image file as metadata stored in private blocks. The set of digital images may further be adapted for storage, transmission, writing, or archiving in a Picture Archive and Communication System (PACS).

According to a further embodiment of the disclosure, a data structure is provided, including redundant symbols contained within the blocks of message symbols such that the number of message blocks is unchanged by the addition of the redundant check symbols. The data structure provides the ability to self-correct missing or corrupted data contained therein. In various embodiments, the data structure 304 may correspond to a single digital image file in a set, or series 300 of digital image files. The data structure may be stored on a computer readable storage medium (described herein below), and may particularly be adapted for storage, display, transmission, retrieval, etc. in a Picture Archive and Communication System (PACS).

The data structure 304 includes a first data field containing a series of image data. The image data may include (m) message symbols. For example, the series of image data may include a plurality of blocks of pixel data. The blocks of pixel data may further include 16 bits per pixel.

The data structure 304 further includes a second field. In certain embodiments in which the data structure comprises a DICOM formatted file, the second data field may be custom tag or field in the DICOM header. In certain embodiments in which the data structure comprises a PNG formatted file, the second data field may be a private block.

The second field includes algorithmically derived check symbols 308 which may be computed based on the (m) message symbols contained in the first field. The algorithmic derivation of the check symbols 308 includes processes as described herein above. The quantity of check symbols 308 may be equal to a quantity (m) of symbols contained in the first data field. In certain embodiments, the second field may further include a final check symbol 310 computed based on the plurality of check symbols. The check symbols 308 and the final check symbol 310 are adapted to protect the data structure from loss in transmission as described herein.

In another embodiment, a data structure may be provided for self-correction of missing or corrupted data contained therein, in which the data structure corresponds to a set 300 of digital image files. The data structure 300 may include a plurality of 2D digital image files that collectively make up a 3D diagnostic imaging scan. Each digital image file may include a first data field containing a series of image data. The image data may include (m) message symbols 302. For example, the series of image data may include a plurality of blocks of pixel data. The blocks of pixel data may further include 16 bits per pixel. The data structure may further include a header, e.g., a DICOM header, containing data relating the particular 2D digital image file to the other digital image files in the set 300. The digital image file may further contain a second data field. In embodiments in which the data structure comprises a DICOM formatted file, the second data field may be custom tag or field in the DICOM header. In embodiments in which the data structure comprises a PNG formatted file, the second data field may be a private block.

In the data structure 300, each digital image file may correspond to a message block 304. As described herein relative to, e.g., FIG. 3, prior to transmission of the data structure 300, the digital image files 304 may be encoded. The second fields of the digital image files 304 may collectively include a plurality of check symbols 308 encoded for a plurality (m) of message symbols 302 contained in the first field in the digital image file or message block 304A . . . 304F. The second fields of the digital image files 304 may further include one or more final check symbols 310 as described herein. The check symbols 308 and final check symbols 310 provide redundancy when distributed across the digital image files 304 in the data structure 300 and are embedded therein in the respective second fields. The data structure may particularly be adapted for archival in a Picture Archive and Communication System (PACS).

Also provided herein is a method of reconstructing a received digital image file in a set of digital image files, wherein the set of digital image files comprises a plurality of check symbols and final check symbols embedded therein. This method is described with reference to FIG. 3, and in particular, processes 5000, 6000, and 7000. These processes may be performed independently of processes 1000, 2000, 3000, and 4000, each of which is previously described.

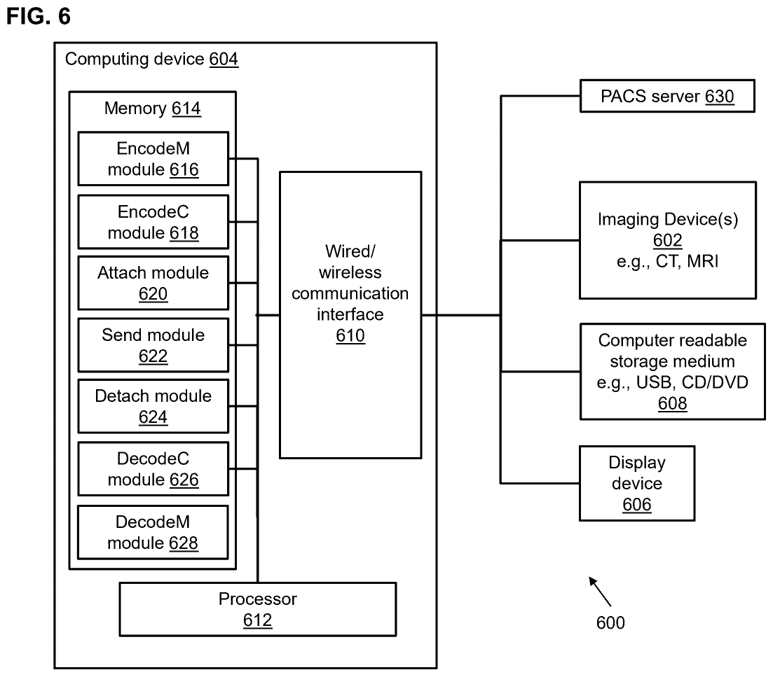

FIG. 6 illustrates a block diagram of a system 600 capable of producing, storing, displaying, transmitting, querying, processing, retrieving, and printing 3D diagnostic imaging scans, in accordance with embodiments of the disclosure. The system 600 may include an imaging device 602 adapted to produce a 3D diagnostic scan of a patient, e.g., using CT, MRI, etc. techniques. The imaging device 602 is in wired or wireless signal communication with a computing device 604, which may be in wired or wireless signal communication with a display device 606 such as, e.g., an LCD or LED-LCD display, and a computer readable storage medium 608 such as, e.g., a USB drive, an optical disc such as a CD or DVD, etc., as enabled by the wired/wireless communication interface 610. Other peripheral devices may also be in wired or wireless signal communication with the computing device such as, e.g., a keyboard, mouse, touchpad, trackball, printer, etc.

The computing device may include a processor 612 configured to execute one or more modules stored in a memory 614 to cause the computing device to perform the processes described herein. These may include, e.g., EncodeM module 616, EncodeC module 618, Attach module 620, Send module 622, Detach module 624, DecodeC module 626, and DecodeM module 628, each of which may be configured to cause the computing device to perform processes 1000, 2000, 3000, 4000, 5000, 6000, and 7000, respectively, as described herein. These processes include the ability to transform a 3D scan made up of a set of 2D digital image files into a set of 2D digital image files containing error correcting code, in order to protect the files from losses, e.g., in transmission of the files, while maintaining compatibility with PACS 630. These processes further include the ability to transmit the set of 2D digital image files to a receiver, to receive the 2D digital image files, and to use the error correcting code embedded within the files to repair or recover any image data that was lost or corrupted.

Computing device 604 is further in wired or wireless signal communication with a PACS server 630. The computing device 604 is adapted to transmit or upload digital image files, such as those acquired via imaging device 602, to the PACS 630. The computing device 604 may also be adapted to receive or download digital image files from the PACS 630, which may include an associated patient image database storing 2D and/or 3D digital image files. Still further, the computing device may be adapted to export 3D scan files, either acquired locally or from PACS 630 to the computer readable storage medium 608.

The PACS 630 may further be in wired or wireless signal communication with a plurality of computing devices (not shown) capable of functions similar to those described herein with respect to computing device 604.

Although the modules 616, 618, 620, 622, 624, 626, and 628 are depicted as residing in the computing device 604, these modules may additionally or alternatively reside in the PACS 630, or another system component in wired or wireless signal communication with the computing device 604 and other devices of the system 600.

In addition, if the set of original image files are not the same size, the processor 612 may be configured to append known symbols (such as all zeros) to selected image files to make them the same size. Also, the image files contain pixel data plus header. Even if the pixel data is the same size, the header sizes could be different. Therefore, padding by the processor 612 will make it possible to use the proposed method by padding either the header or the pixel data or both.

Example embodiments are described herein with reference to block diagrams and/or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks. Accordingly, embodiments of present inventive concepts may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated, and/or blocks/operations may be omitted without departing from the scope of inventive concepts. Moreover, although some of the diagrams include arrows on communication paths to show a primary direction of communication, it is to be understood that communication may occur in the opposite direction to the depicted arrows.

As used herein, the terms “first,” “second,” and the like, do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by the context (e.g., includes the degree of error associated with measurement of the particular quantity). The suffix “(s)” as used herein is intended to include both the singular and the plural of the term that it modifies, thereby including one or more of that term (e.g., the metal(s) includes one or more metals). Ranges disclosed herein are inclusive and independently combinable (e.g., ranges of “up to about 25 mm, or, more specifically, about 5 mm to about 20 mm,” is inclusive of the endpoints and all intermediate values of the ranges of “about 5 mm to about 25 mm,” etc.).

While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method for protecting a set of digital image files from loss, wherein the set of digital image files comprises a plurality of digital image files of a same size, the method comprising:
encoding each digital image file in the plurality of digital image files by:
computing a plurality of check symbols for a plurality of symbols contained in a same location in each digital image file; and
generating a plurality of check blocks, each check block in the plurality thereof including a quantity of check symbols equal to a number of symbols contained in the digital image file;
encoding each check block in the plurality thereof by computing a final check symbol therefor; and
embedding the check symbols and the final check symbols into the plurality of digital image files, wherein the check symbols and the final check symbols are distributed approximately evenly over the plurality of digital image files.

2. The method of claim 1, further comprising transmitting the set of digital image files to a receiver.

3. The method of claim 2, further comprising, after the transmitting:
decoding each digital image file in the received set of digital image files by:
detaching the check symbols and the final check symbols from the plurality of digital image files;
creating a plurality of error-correction blocks using the detached check symbols and final check symbols; and
performing error-correction by:
recovering or repairing one or more received error-correction blocks; and
using the recovered or repaired error-correction blocks to recover or repair received digital image files.

4. The method of claim 1, further comprising:
after the embedding, encoding each digital image file having the check symbol embedded therein, to add a secondary check symbol within each digital image file.

5. The method of claim 3, further comprising:
after the detaching, and prior to the creating, decoding the secondary check symbol within each digital image file.

6. The method of claim 1, wherein the embedding comprises an iterative series of embedding operations.

7. The method of claim 1, wherein each digital image file in the plurality thereof comprises Digital Imaging and Communications in Medicine (DICOM) formatting.

8. The method of claim 7, wherein each digital image file in the plurality thereof comprises a block of pixel data and a DICOM header, and wherein the embedding comprises embedding the check symbols and the final check symbol into the DICOM header as metadata.

9. The method of claim 1, wherein the plurality of digital image files comprises a series of portable network graphics (PNG) image files, and wherein the embedding comprises embedding the check symbols and the final check symbol into the PNG image files as private blocks.

10. The method of claim 1, wherein the set of digital image files are adapted for at least one of:
storage, transmission, writing, or archiving in a Picture Archive and Communication System (PACS).

11. The method of claim 1, wherein the set of digital image files represents a computed tomography (CT) scan or a magnetic resonance imaging (MRI) scan.

12. A data structure stored on a computer readable medium comprising:
a set of data sub-structures, wherein each data sub-structure corresponds to a digital image, and the set of digital images collectively corresponds to a three-dimensional (3D) scanned volume, wherein each data sub-structure in the set thereof comprises:
a first data field containing a series of image data; and
a second data field containing a plurality of check symbols computed based on the series of image data contained in the first data field, wherein a quantity of check symbols contained in the second data field is equal to a quantity of image data symbols contained in the first data field, and
wherein the plurality of check symbols are adapted for decoding by a processor to protect the data structure from loss in transmission.

13. The data structure of claim 12, wherein each data sub-structure includes Digital Imaging and Communications in Medicines (DICOM) formatting, wherein the second data field comprises a custom tag in a DICOM header.

14. The data structure of claim 12, wherein each data sub-structure includes Portable Network Graphics (PNG) formatting, wherein the second data field comprises a private block.

15. The data structure of claim 12, wherein the series of image data comprises a plurality of blocks of pixel data.

16. The data structure of claim 15, wherein the pixel data comprises 16 bits per pixel.

17. The data structure of claim 12, being adapted for archival in a Picture Archive and Communication System (PACS).

18. The data structure of claim 12, wherein each digital image is a two-dimensional (2D) slice of the 3D scanned volume, and wherein the 3D scanned volume comprises a computed tomography (CT) or magnetic resonance imaging (MRI) scan.

19. A method of reconstructing a digital image file in a set of digital image files, wherein the set of digital image files comprises a plurality of check symbols and final check symbols embedded therein, the method comprising:
detaching the check symbols and the final check symbols from the set of digital image files;
creating a plurality of error-correction blocks using the detached check symbols and final check symbols; and
reconstructing the digital image file by:
recovering or repairing one or more error-correction blocks; and
using the recovered or repaired error-correction blocks to reconstruct the digital image file.

20. The method of claim 18, wherein the set of digital image files represents a computed tomography (CT) scan or a magnetic resonance imaging (MRI) scan.
